(19) United States
(12) Patent Application Publication
(10) Pub. No.: US 2012/0276154 A1
(43) Pub. Date: Nov. 1, 2012

(54) CREATION OF HAIR FOLLICLES IN TISSUE-ENGINEERED SKIN GRAFTS

(76) Inventors: Seyed Babak Mahjour, Holmdel, NJ (US); Hongjun Wang, Millburn, NJ (US)

(21) Appl. No.: 13/455,649

(22) Filed: Apr. 25, 2012

Related U.S. Application Data
(60) Provisional application No. 61/478,983, filed on Apr. 26, 2011.

(51) Int. Cl. A61F 2/10; A61P 17/00; A61K 35/36

(52) U.S. Cl. 424/400; 424/93.7

(57) ABSTRACT

A living tissue-engineered skin graft having a potential to develop hair follicles includes a multilayered skin-like structure of alternating nanofiber mats and layers of fibroblasts assembled in a layer-by-layer fashion. Aggregates of dermal papilla capable of developing into hair follicles are embedded in the multilayered structure such that the aggregates develop into hair follicles upon culturing the multilayered structure. Keratinocytes are provided as an outer layer of the skin graft. Fibroblasts, keratinocytes and dermal papilla cells are isolated from skin and cultured to form suspensions of cells for fabricating the skin graft. Aggregates of dermal papilla cells are generated using a hanging drop method. Nanofiber mats are formed by electrospinning biocompatible materials onto a culture media or a layer of fibroblast suspension. The skin graft has dermal and epidermal layers and provides a biomimetic environment to promote healing and hair growth.

### CROSS-REFERENCE TO RELATED APPLICATIONS

The present application claims the benefit of U.S. Provisional Patent Application No. 61/478,983, filed on Apr. 26, 2011, which is incorporated by reference herein in its entirety.

### STATEMENT REGARDING FEDERALLY SPONSORED RESEARCH

Not applicable.

### FIELD OF THE INVENTION

The present invention relates to a tissue-engineered skin graft, and more particularly to a tissue-engineered skin graft with hair follicles.

### BACKGROUND OF THE INVENTION

Current tissue-engineered skin grafts have been recognized as a promising treatment for chronic ulcers and acute burns, leading to a rapid closure of wounds to prevent further dehydration and potential infection. However, known grafts fail to regenerate many essential skin structures such as hair, nerves, vessels and glands. The lack of hair in healed wounds not only fails to provide the necessary physiologic protection to skin, but also psychosocially impacts an individual's self-esteem and interpersonal relationships within a society. Extensive efforts have been made to reconstitute hair follicles, but mainly focus on in vivo regeneration.

Hair follicles are complex miniorgans with numerous functions including production of hair shafts, acting as a sensory instrument and serving as a psychosocial communication tool symbolically representing youth, health, and fertility. Hair undergoes cyclical growth patterns through the stages of anagen (rapid growth), telogen (quiescence), and catagen (regression). This growth cycle provides a mechanism for cleaning skin debris, parasites, and harmful chemicals by encapsulating them within trichocytes. It also protects rapidly dividing keratinocytes from malignant degeneration and oxidative damage. In addition to production of keratins and melanin for the hair shaft, hair follicles produce a wide array of hormones, neurotransmitters, neuropeptides, and growth factors. Many of these growth factors, such as the molecules FGF, EGF, IGF, HGF, TGF-beta, VEGF, and NGF, are known for their crucial roles in wound healing and skin homeostasis. Apart from its clinical importance, the hair follicle offers an easily manipulated, widely available test system for many areas of general biology including differentiation, proliferation, apoptosis, stem cell biology, extracellular matrix remodeling, immune defense, and immune privilege.

### SUMMARY OF THE INVENTION

In one aspect, the present invention provides a tissue-engineered skin graft capable of developing hair follicles. In some embodiments of the present invention, the skin graft includes multiple alternating mats of biocompatible nanofibers and layers of skin cells. In some such embodiments, the skin cells include cultured fibroblasts. In some such embodiments, the skin cells include aggregates of dermal papilla cells (dermal papilla aggregates) capable of differentiating into hair follicles. In some such embodiments, the dermal papilla aggregates are embedded within the skin graft in a predetermined arrangement. In some such embodiments, the dermal papilla aggregates are embedded within the skin graft to provide the skin graft with predictable hair follicle and hair formation capabilities. In some embodiments, the outer layers of the skin graft include layers of epidermal keratinocytes. In some such embodiments, the skin graft has distinguishable dermal and epidermal sections. In some embodiments, the skin graft provides a biomimetic environment for interactions between the dermis, epidermis, hair follicles and proto hairs. In some such embodiments, the nanofiber mats include biodegradable or bioresorbable materials. In some such embodiments, the biodegradable or bioresorbable materials include synthetic polymers, natural polymers, or blends thereof. In some such embodiments, the biodegradable or bioresorbable materials include polycaprolactone, collagen, or blends thereof. In some such embodiments, different layers of nanofibers have different chemical compositions. In some embodiments, the nanofiber layers include substances such as drugs or growth factors to be released in situ after transplantation of the skin graft into a patient.

In another aspect, the present invention provides a method for making a tissue-engineered skin graft capable of developing hair follicles. In some embodiments, the method includes, but is not limited to, the steps of: (1) isolating skin and hair-follicle forming cells (e.g., dermal papilla cells) from intact skin and culturing the isolated cells for assembly into tissue-engineered skin grafts; (2) formation of dermal papilla aggregrates by culturing the isolated dermal papilla cells; and (3) layer-by-layer assembly of cultured skin cells and dermal papilla aggregates with alternating layers of biocompatible nanofiber mats so as to form three-dimensional constructs. In some such embodiments, the three-dimensional constructs are further cultured to differentiate the dermal papilla aggregrates into hair follicles and produce proto hairs. In some such embodiments, the dermal papilla aggregates are cultured by a hanging drop method. In some such embodiments, the method includes building up alternating layers of nanofibers and fibroblasts, adding a layer of a dermal papilla aggregates, adding further alternating layers of nanofibers and fibroblasts, and adding layers of keratinocytes as an epidermal layer. In some such embodiments, dermal papilla aggregates are selectively deposited where hair growth is desired. In some such embodiments, the skin structure is formed so as to have distinct dermal and epidermal layers.

### BRIEF DESCRIPTION OF THE DRAWINGS

For a more complete understanding of the present invention, reference is made to the following detailed description of exemplary embodiments thereof, considered in conjunction with the accompanying drawings, in which:

FIG. 1 is a schematic diagram of an exemplary skin graft according to an embodiment of the present invention;

FIG. 2 is a microphotograph of keratinocytes obtained and cultured by an exemplary method according to an embodiment of the present invention;

FIG. 3 is a microphotograph of follicular cells (i.e., dermal papilla cells) obtained and cultured by an exemplary method according to an embodiment of the present invention;

FIG. 4 is a microphotograph of fibroblasts obtained and cultured by an exemplary method according to an embodiment of the present invention;

FIG. 5 is a microphotograph of dermal papilla cells stained to indicate the presence of α-smooth muscle actin;

FIG. 6 is a microphotograph of dermal papilla aggregates prepared by an exemplary method according to an embodiment of the present invention and incubated for 18 hours;

FIG. 7 is a microphotograph of dermal papilla aggregates prepared by an exemplary method according to an embodiment of the present invention and incubated for 2 days;

FIG. 8 is a microphotograph of dermal papilla aggregates prepared by an exemplary method according to an embodiment of the present invention and incubated for 5 days;

FIG. 9 is a microphotograph of a dermal papilla aggregate prepared by an exemplary method according to an embodiment of the present invention with an initial inoculum of 20,000 dermal papilla cells;

FIG. 10 is a microphotograph of a dermal papilla aggregate prepared by an exemplary method according to an embodiment of the present invention with an initial inoculum of 40,000 dermal papilla cells;

FIG. 11 is a schematic drawing illustrating a method of preparing a mat of nanofibers by electrospinning according to an embodiment of the present invention, with an inset microphotograph of such a nanofiber mat;

FIG. 12 is a schematic drawing illustrating a method of adding cells to the mat of FIG. 11 according to an embodiment of the present invention;

FIG. 13 is a schematic drawing illustrating the method of FIG. 12 extended to forming a cell-seeded layer on the mat of FIGS. 11 and 12;

FIG. 14 is a schematic drawing of a skin graft made according to embodiments of the methods of FIGS. 11-13;

FIG. 15 is a microphotograph of a stained thin section of a skin graft prepared by an exemplary method according to an embodiment of the present invention;

FIG. 16 is a microphotograph of another stained thin section of a skin graft prepared by an exemplary method according to an embodiment of the present invention;

FIG. 17 is a microphotograph of a stained thin section of a skin graft according to an embodiment of the present invention after incubation; and

FIG. 18 is an enlargement of a portion of FIG. 17.

### DETAILED DESCRIPTION OF THE INVENTION

The present invention provides tissue-engineered skin grafts having hair follicles capable of providing new hair and methods of making such skin grafts. These skin grafts are prepared in vitro using a biomimetic approach to generating hair follicle-like structures directly in tissue-engineered skin grafts. In embodiments of the present invention, the biomimetic approach involves three major types of cells, including hair-forming cells (i.e., dermal papilla) and cells from the inner and outermost layers of skin (i.e., dermal fibroblasts and keratinocytes), which may be autologous or allogenic with regard to the subject receiving the skin graft. In some embodiments of the present invention, the dermal papilla (“DP”) cell aggregates may be made by a hanging droplet method, and assembled with the skin cells into three-dimensional (“3D”) skin substitutes with the assistance of biocompatible nanofibers, such as polycaprolactone (PCL)/collagen-blended electrospun nanofibers, following a layer-by-layer assembly approach. The 3D skin substitutes develop hair follicle-like structures in vitro (“proto hair”), and consequently form new hairs in vivo after transplantation to full-thickness skin wounds. Compared to conventional in vivo injection of hair cells, the inventive process provides better control of the number of hair follicles formed within a specific area of skin, and at specified locations in the skin, by embedding various amount of DP aggregates at specified locations within the skin grafts. In addition, hair follicle size can be controlled by controlling the size of the DP aggregates. For those deep wounds like third degree skin burns, which lose epidermis and dermis portions, injection of hair cells cannot make any new hair shafts because of the lack of dermal-epidermal interaction. As a result of the presence of hair structures in tissue-engineered skin grafts, the wound-healing process can be accelerated by transplanting such grafts to the wounded area. Further, the tissue-engineered skin grafts provide the potential for new hair shafts to form.

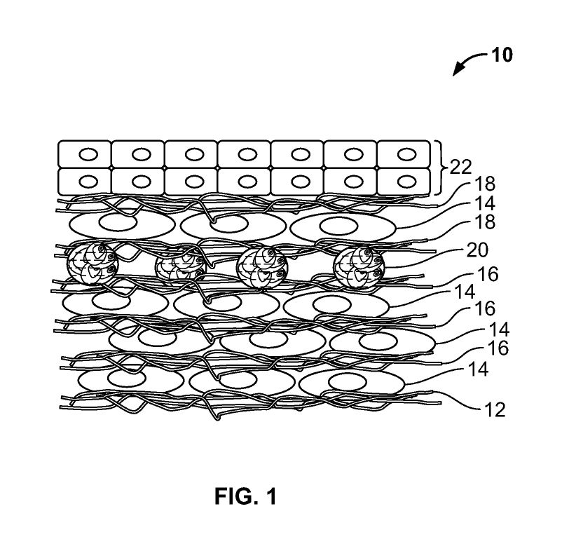

Referring to an exemplary tissue-engineered skin graft 10 of FIG. 1, an embodiment of a tissue-engineered skin graft according to the present invention includes, from the bottom upward: a basement of electrospun biocompatible nanofibers 12, alternating layers of fibroblasts 14 and electrospun biocompatible nanofibers 16, 18, DP aggregates 20 between layers of electrospun biocompatible nanofibers 18, and, as uppermost layers, layers of epidermal keratinocytes 20. Electrospun biocompatible nanofibers 16, 18 may be formed from different materials than each other, have different fiber densities, or other differences to impart particular mechanical or biological properties to the tissue-engineered skin graft 10. Suitable materials for the nanofibers include synthetic polymers, natural polymers, or blends thereof. Suitable synthetic polymers include, but are not limited to, polycaprolactone, polylactic acid, polyglycolic acid, poly(lactic-co-glycolic acid), and blends thereof. Suitable natural polymers include, but are not limited to, collagen, elastin, fibrinogen, glycosaminoglycans, or blends thereof. Other substances, such as drugs, medicaments, or growth factors, may be blended into the nanofibers to aid in healing or regrowth of tissues, or to encourage formation of hair follicles or hair growth.

An exemplary method of forming a tissue-engineered skin graft, such as skin graft 10 of FIG. 1, according to the present invention includes, but is not limited to, the following steps:

Step 1: Isolation of skin and hair follicle-forming cells. Three major types of cells (dermal papillae, dermal fibroblast and epidermal keratinocyte) are isolated by enzymatic methods and cultured in vitro to obtain cells needed to construct the skin graft 10. DP cells are isolated from intact hair follicles and keratinocyte and fibroblasts are isolated from intact skin.

Step 2: Formation of dermal papilla aggregates. DP aggregates are formed from the DP cells obtained in step 1 using an adaptation of a hanging droplet method.

Step 3: Layer-by-layer assembly of skin cells and DP aggregates into 3D constructs with formation of hair follicle-like structures. PCL/collagen nanofibers are used to assist the assembly of skin cells (dermal fibroblast and keratinocyte) together with DP aggregates into 3D skin substitutes, and the formed constructs are further cultured in 5% CO₂ and 37° C. for 14 days to form the proto hair-like structures.

Representative methods useful for step 1 may be found in the following references, each of which is incorporated by reference herein in its entirety: (Ref. 1) Chiu, H. C., et al. An efficient method for isolation of hair papilla and follicle epithelium from human scalp specimens. Br J Dermatol. (1993) 129:350-351; (Ref. 2) Wu, J. J., et al. Enzyme digestion to isolate and culture human scalp dermal papilla cells: a more efficient method. Arch Dermatol Res. (2005) 297:60-67; (Ref. 3) Li, Y., et al. One-step collagenase I treatment: an efficient way for isolation and cultivation of human scalp dermal papilla cells. J Dermatol Sci. 2005; 37: 58-60; (Ref. 4) Vaughan, F. L., et al. Isolation, Purification, and Cultivation of Murine and Human Keratinocytes. Methods in Molecular Biology. (2005) 290:187-206; and (Ref. 5) Wang, H., et al. Improved enzymatic isolation of fibroblasts for the creation of autologous skin substitutes. In Vitro Cellular & Developmental Biology—Animal (2004) 40 (8 & 9): 268-277. Representative methods useful for step 2 may be found in the following reference, which is incorporated by reference herein in its entirety: (Ref. 6) Qiao, J., et al. Hair morphogenesis in vitro: Formation of structures suitable for implantation. Regen. Med. (2008) 3 (5), 683-692. Representative methods useful for step 3 may be found in the following reference, which is incorporated by reference herein in its entirety: (Ref. 7) Yang, X, et al. Nanofiber enabled layer-by-layer approach toward three-dimensional tissue formation. Tissue Engineering Part A. (2009) 15: 945. Certain applications of the aforesaid methods are discussed more fully hereinbelow.

To demonstrate the feasibility of an embodiment of the present invention, the prototype study described hereinbelow was performed using rat skin cells and rat hair follicle cells. Rat cells were selected as models for the embodiment because of similarities between the growth cycles of rat hair and those of human hair. The prototype study is discussed in the following Example 1. The following example is presented to illustrate certain embodiments of the present invention, and is not intended to limit the scope of the invention in any way.

### EXAMPLE 1

The present Example 1 employs steps 1-3 of the exemplary method discussed above with respect to an embodiment of the present embodiment to construct an exemplary skin graft, such as skin graft 10 of FIG. 1. The skin graft itself is also discussed hereinbelow.

### Step 1: Isolation of Skin and Hair Follicle-Forming Cells.

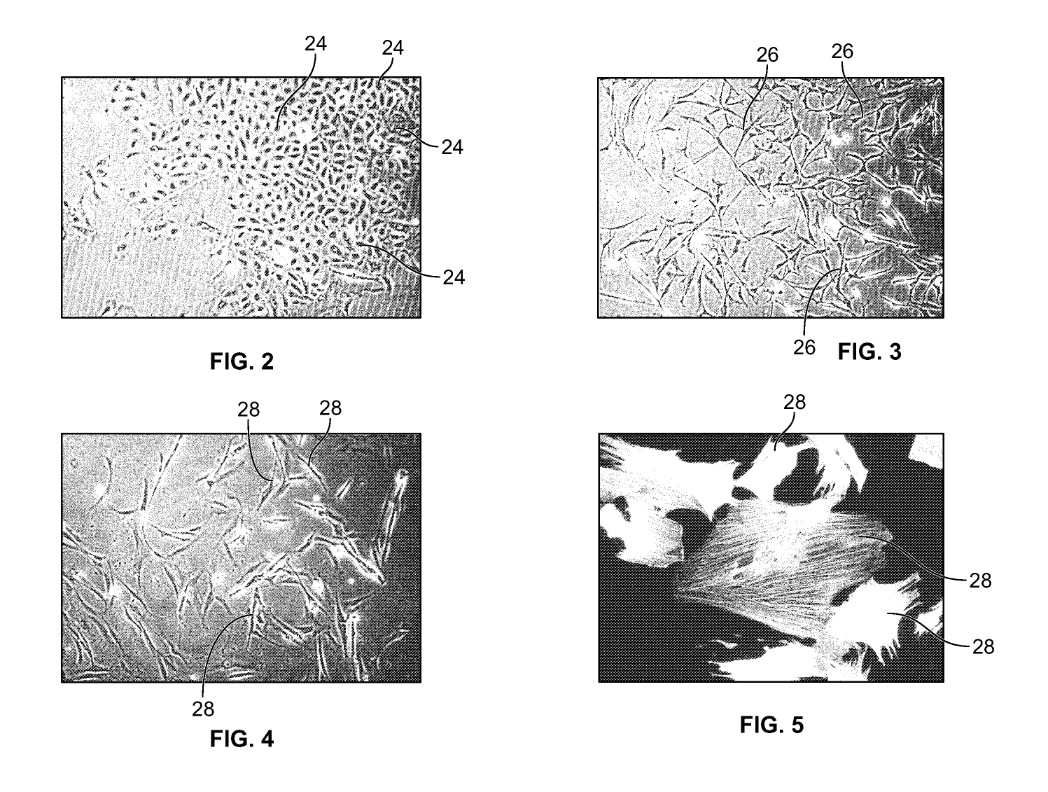

Enzymatic dissociation methods employing collagenase I and dispase II were optimized and established for obtaining keratinocytes from skin epidermis and DP cells from vibrissa hair follicles of the same female pregnant Sprague Dawley rat. To circumvent the relatively low yield and limited proliferation capacity of fibroblasts isolated from rat skin, fibroblasts were isolated from a fetus of the same female rat. FIGS. 1-3 show the typical morphology of all three types of cells: keratinocytes 24, which are positive for “keratin 14” (“K14”) (FIG. 1), fibroblasts 26, which are positive for vimentin (FIG. 2), and DP cells 28, (FIG. 3), which are positive for α-smooth muscle actin (α-SMA) (FIG. 5). α-SMA is recognized as a reliable marker for DP cells, greater than 95% have been shown to test positive for this marker.

Follicular cells (i.e., DP cells), keratinocytes and fibroblasts were isolated using the established protocols of the References 1-5 that are identified above. DP cells were isolated from rat vibrissae using methods from References 1-3. References 4 and 5 were used as guidance to isolate keratinocytes from the skin epidermal layer and fibroblasts from fetal skin.

In brief, a pregnant, female Sprague-Dawley rat with 15-19 day old fetuses was sacrificed. Under a dissecting microscope, the hair bulbs were isolated from the hair follicles of the adult rat. The hair bulbs thus obtained were then digested in a collagenase I solution [1 mg/mL] at 37° C. to release the DP cells. The collected DP cells were then cultured in Dulbecco's Modified Eagle Medium (DMEM), containing b-FGF at 10 ng/mL, 14% fetal bovine serum (“FBS”), and 1% penicillin/streptomycin, yielding cultured DP cells such as DP cells 28 of FIG. 3.

Keratinocytes were isolated from adult rat dorsal skin as described herein, using methods described in References 4 and 5 with modifications to the enzymatic digestion steps. The adult rat was shaved, and then a 3×3 cm area of bare skin was harvested, and incubated in a dispase II solution (0.25% w/v) for 30-60 min to separate the epidermis from dermis. The detached epidermis was then incubated in 0.05% trypsin/EDTA to release keratinocytes. The keratinocytes were then cultured in keratinocyte serum-free medium (KSFM, Invitrogen™, Life Technologies Corporation, Grand Island, N.Y.), yielding cultured keratinocytes such as the keratinocytes 24 of FIG. 2.

Fibroblasts were obtained following the same procedure as in Reference 5. Fetal skin was harvested and the dermis was minced and then digested in 0.25% dispase II and 0.75% collagenase I for 1.5-2 hours. After centrifuging, the fibroblasts thus obtained were cultured in DMEM with 10% FBS, and 1% penicillin/streptomycin. Culture medium was refreshed every 2-3 days, yielding cultured fibroblasts, such as fibroblasts 26 of FIG. 3.

### Step 2: Formation of Dermal Papilla Aggregates.

DP aggregates were generated using a modification of the hanging droplet method of Reference 6, identified above. Briefly, the isolated DP cells obtained in step 1 were suspended in DMEM with high glucose, 14% FBS, and 0.24% methyl cellulose. The cell suspension was titered on the bottom of a 100-mm Petri dish as 20 μL droplets (each droplet containing about 3×10⁴ cells). The Petri dish was then inverted, so that the droplets were suspended from the Petri dish. The suspended droplets were incubated at 37° C. in a 5% CO₂ atmosphere. DP aggregates formed within 18-20 h. Upon formation, the DP aggregates were individually transferred to round-bottom 96-well plates with low-ultra retention to prevent DP aggregates from adhering to the plate. The culture medium was changed every 2-3 days.

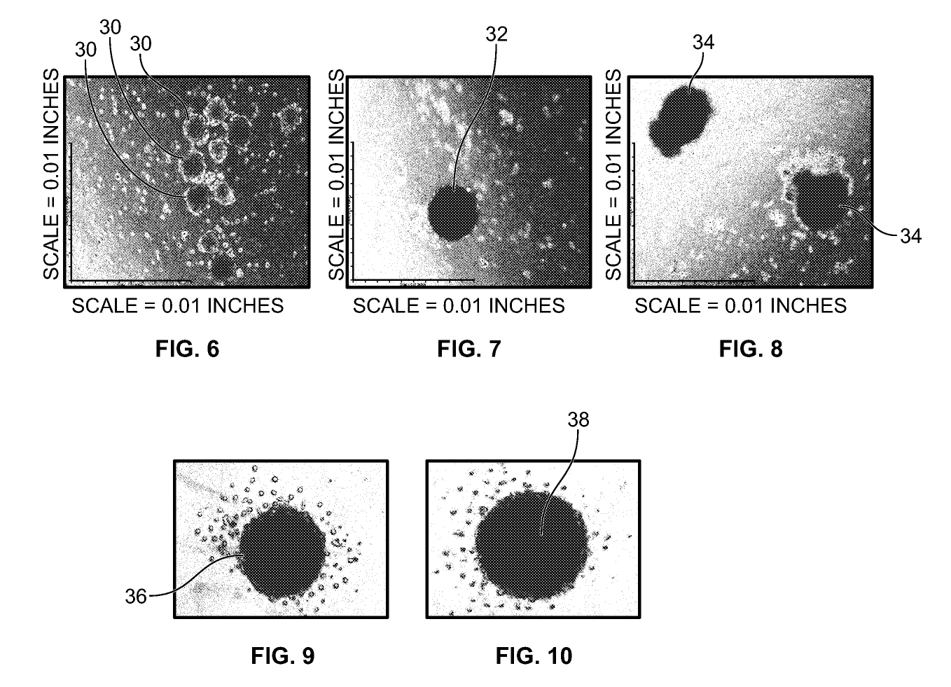

FIGS. 6-8 are microphotographs presenting the size of DP aggregates produced by the above method at 18 hours (DP aggregates 30), 2 days (DP aggregate 32) and 5 days (DP aggregates 34), respectively. The sizes of the DP aggregates remained stable and by day 5 the DP cells began to grow outward (see, e.g., FIG. 8). Sizes of the DP aggregates were uniform, controlled by the number of cells deposited in each droplet. For example, the DP aggregates 36, 38 of FIGS. 9 and 10 (shown at the same scale) initially contained 20,000 and 40,000 cells, respectively. The DP aggregate 38 of FIG. 10 is clearly larger than DP aggregate 36 of FIG. 9. It was observed that interior cells of larger aggregates (e.g., aggregates of about 400-500 μm in diameter (not shown)) could not survive in long-term cultures, probably due to insufficient oxygen and nutrient at the center of the aggregates.

Step 3: Layer-by-Layer Assembly of Skin Cells and DP Aggregates into 3D Constructs with Formation of Hair Follicle-Like Structures.

To rapidly form skin grafts with spatially-controlled cell distribution (e.g., the distribution of DP aggregates), the layering method of Reference 7 was modified to enable layer-by-layer assembly of an exemplary skin graft having proto-hair follicles. The use of cell layering in combination with the in situ electrospinning of biocompatible nanofibers, enables the maintenance of hydrated cells and nanofibers, better control of cell types and cell seeding density and distribution, and, perhaps most importantly, incorporation of the cells in a 3D biomimetic environment for better expression of their phenotypes.

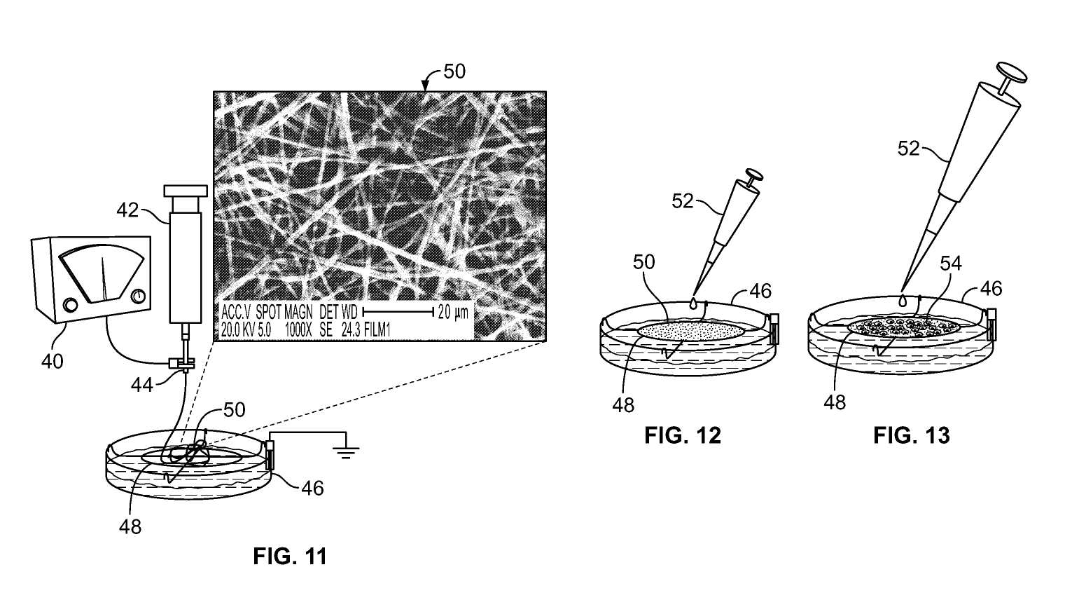

Referring to FIGS. 11-14, skin grafts having hair follicles were fabricated by assembling DP aggregates together with skin cells and nanofibers into 3D structures using a layer-by-layer assembly technique. Briefly, referring to FIG. 11, a high voltage source 40 was used to apply a voltage in the range of about 10-15 kV to an electrospinning device 42, described in Reference 7 and known in the tissue-engineering arts, between the tip-blunt needle 44 and an electrically-grounded culture medium 46 (DMEM with 14% FBS). To assist cell seeding and define the shape of the multilayered cell/fiber construct, a stainless steel wire loop 48 (3 cm in diameter) was positioned in the culture medium 46.

Using this modified approach, constructs with both fibroblasts and keratinocytes (bilayer skin constructs) were created. Referring first to FIG. 11, a layer of collagen/PCL nanofibers 50 was first electrospun onto the culture medium 46, and then 1 mL of fibroblast suspension (1×10⁶ cells/mL) (represented by applicator 52 of FIG. 12) was evenly seeded onto the fiber mesh 50, forming a cell-seeded layer 54, (see FIG. 13). Then, a second layer of collagen/PCL nanofibers (not shown) was electrospun onto the cell-seeded layer 54 as described with respect to FIG. 11. By repeating the above steps of FIGS. 11-13, alternating-multilayered constructs were formed, including eight layers containing fibroblasts and the two outermost layers of keratinocytes. After the nanofiber mats and layers of fibroblasts were laid down, DP aggregates were seeded and the layer-by-layer build-up continued with two layers of keratinocyte seeding. The multilayered constructs were further cultured in a humidified incubator at 37° C. with 5% CO₂ for up to 14 days. Culture medium was refreshed every 2-3 days.

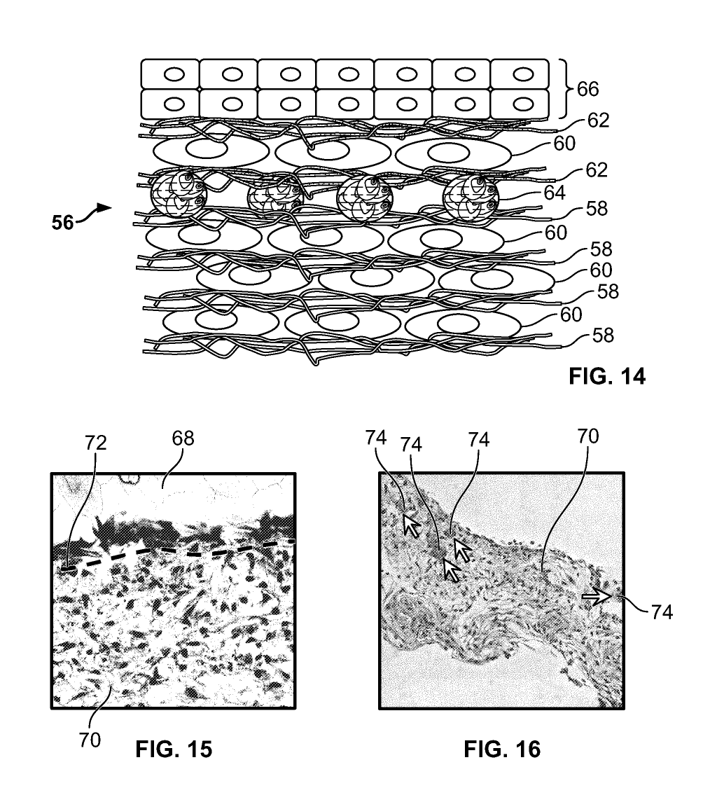

A simplified schematic of such a construct 56 is shown as FIG. 14, wherein: nanofibers having a first composition are shown as layers of nanofibers 58; fibroblasts are shown as layers of fibroblasts 60 (with the uppermost layer representing three layers of fibroblasts); nanofibers having (optionally) a second composition are shown as a layer of nanofibers 62; DP aggregates are shown as a layer of DP aggregates 64; and keratinocytes are shown as a double-layer of keratinocytes 66. In an exemplary embodiment of the skin substitute according to the present invention the nanofibers of layers 58 may be PCL/collagen in a ratio of 3:1, and the nanofiber of the layers 62 may be PCL/collagen in a ratio of 1:1. One having ordinary skill in tissue-engineering will recognize other suitable materials for the various nanofibers of the skin substitute of FIG. 14, which may include those discussed elsewhere herein.

Rat Skin Grafts with Hair Follicle Structures.

The layer-by-layer cell assembly approach discussed above was used to create a bi-layer skin substitute with DP aggregates entrapped in the interface between fibroblasts and keratinocytes. A simplified schematic of a prototype of such a skin substitute was presented in FIG. 14. FIGS. 15 and 16 are microphotographs of stained thin sections of the skin substitute. FIG. 15 shows that the skin substitute 56 has both epidermal 68 and dermal 70 layers. A boundary 72 between the layers 68, 70 is indicated by a dashed line. FIG. 16 shown DP aggregates 74 (also indicated by white arrows) in the dermal layer 70.

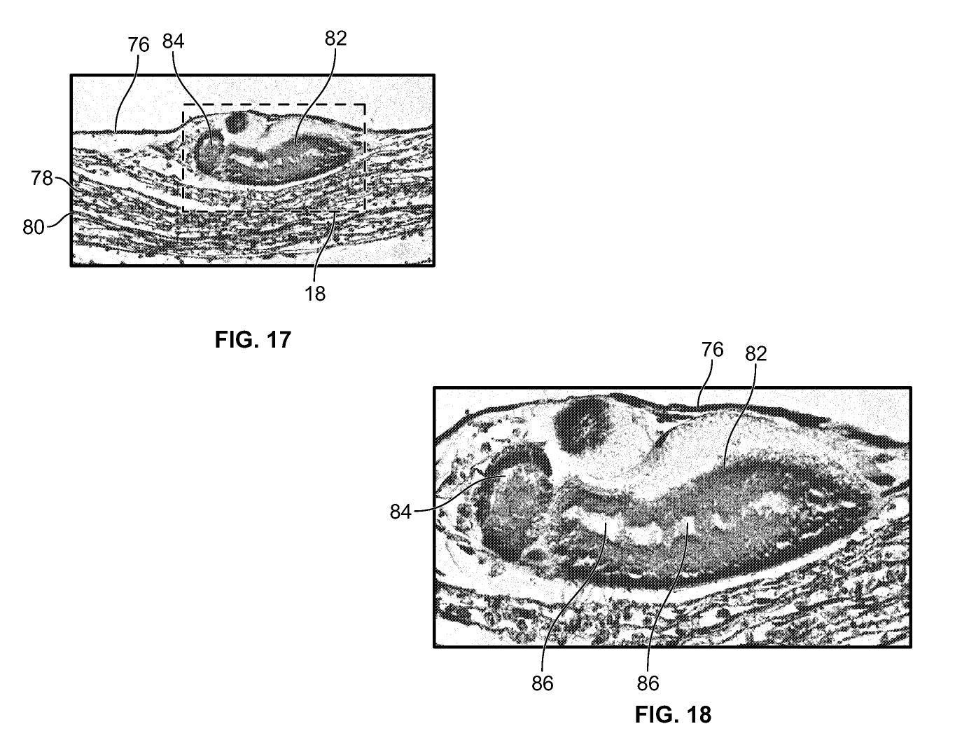

It was also found that proto hair was formed in the skin substitute after being cultured for 14 days. After being cultured for two weeks, thin cross-sections of cultured skin substitute were stained with hematoxylin and eosin (H&E). Referring to FIG. 17, keratin 76, fibroblasts 78, and nanofiber layers 80 can be observed. Further, a proto hair structure 82 can be observed, having a round end 84 that has the appearance of a DP aggregate. FIG. 18 is an enlarged view of the proto hair of FIG. 17, showing a partially keratinized proto hair shaft 86, similar to a hair follicle. This result shows that DP aggregates can develop into hair follicle-like structures in skin grafts.

The exemplary embodiments of the present invention allow the use the inventive skin graft for hair regeneration and wound healing in one transplantation. Wound healing is accelerated by the presence of the hair follicles. The presence of dermal-epidermal interaction can accelerate the hair regeneration. The density, size and location of the hair follicles can be controlled in the skin grafts.

The skin graft and method of its making are also cost-effective, in that the average cost for hair transplantation under current practices (April 2012) can be in the range of from $2,500 to $9,000 for a 5×5 cm skin graft and individual hair implantation costs of $3 to $8. In contrast, the expected cost of transplanting a 5×5 cm skin graft of the present invention, which includes hair follicles grown in the graft, would be less than about $1,000.

It will be understood that the embodiment described herein is merely exemplary and that a person skilled in the art may make many variations and modifications thereto without departing from the spirit and scope of the invention. All such variations and modifications are intended to be included within the scope of the invention described in the claims appended hereto.

We claim:

1. A living tissue-engineered skin graft capable of developing hair follicles, comprising:
a plurality of mats including nanofibers of at least one biocompatible material;
a plurality of layers of viable fibroblasts, each of said layers of viable fibroblasts covering at least a portion of a corresponding one of said plurality of mats;
a plurality of aggregates of viable dermal papilla cells, said aggregates having a potential to develop into hair follicles within said skin graft; and
a plurality of viable keratinocytes, wherein said mats and said layers of viable fibroblasts are arranged in a multilayered structure having alternating ones of said mats and said layers of viable fibroblasts and said keratinocytes are arranged on one of said mats such that some of said keratinocytes are an outer layer of said skin graft, and wherein said aggregrates of viable dermal papilla cells are embedded in said multilayered structure such as to allow said aggregates of dermal papilla cells to develop into hair follicles.

2. A method of preparing a living tissue-engineered skin graft capable of developing hair follicles, said method comprising the steps of:
isolating viable dermal papilla cells, fibroblasts, and keratinocytes from one or more samples of skin;
culturing the fibroblasts to produce a plurality of viable fibroblasts and preparing a suspension thereof;
culturing the keratinocytes to produce a plurality of viable keratinocytes and preparing a suspension thereof;
preparing aggregates of the dermal papilla cells following a hanging drop protocol that includes the steps of suspending the isolated dermal papilla cells in a growth-supporting media to form a suspension of the isolated dermal papilla cells, titering drops of the suspension of the isolated dermal papilla cells onto a substrate such that each of the drops contains a selected number of dermal papilla cells, inverting the substrate such that the drops are suspended from the substrate, incubating the drops on the inverted substrate, thereby forming aggregates of dermal papilla cells within the drops, and culturing the aggregates of dermal papilla cells;
producing a mat of biocompatible nanofibers on a culture medium using an electrospinning technique and depositing a layer of the fibroblast suspension on the mat;
producing another mat of biocompatible nanofibers on the layer of the fibroblast suspension using an electrospinning technique and depositing another layer of the fibroblast suspension on the another mat;
repeatedly producing mats of biocompatible nanofibers and layers of the fibroblast suspension in a layer-by-layer assembly approach, thereby producing a multilayered structure of alternating mats and layers of the fibroblast suspension;
depositing the cultured aggregates of dermal papilla cells in the multilayered structure, whereby the cultured aggregrates of dermal papilla cells are embedded in the multilayered structure so as to allow the aggregates of dermal papilla cells to develop into hair follicles.
